(19) United States
(12) Patent Application Publication
(10) Pub. No.: US 2013/0282392 A1
(43) Pub. Date: Oct. 24, 2013

(54) SYSTEM AND METHOD OF SECURELY STORING, DISPENSING, AND INVENTORYING MEDICATIONS AND SAMPLES

(71) Applicant: Scott Wurm, Celebration, FL (US)

(72) Inventor: Scott Wurm, Celebration, FL (US)

(21) Appl. No.: 13/452,520

(22) Filed: Apr. 20, 2012

(51) Int. Cl. G06Q 50/22

(52) U.S. Cl. 705/2

(57) ABSTRACT

A system and a method of securely storing, dispensing and inventorying medications using secured cabinets, a communication link and a central management server. Each cabinet has compartments to hold prescribed medical items or samples, restricts access to pre-registered users, keeps track of amount of medical items withdrawn by a user for a given patient and how much of the medical items are remaining in the compartments. Video cameras record the transactions. The user, patient and medical item information is transmitted over a communication link to a central management server which keeps records of the withdrawn medical items and corresponding information, determines when compartments of the cabinets need refilling, and provides billing information for a customer and for specific patients. The central management server is also able to keep track of samples provided in the cabinets and provide advertising on GUIs of the cabinets and in waiting areas of customers.

### BACKGROUND OF THE INVENTION

1. Field of the Invention

There is a strong need for a system and method of dispensing medications, and more particularly, to an automated system and method for securely storing, dispensing, and inventorying controlled medications and samples.

2. Description of the Related Art

Today, there are approximately 5,800 hospitals, 5,000 ambulatory surgery centers (ASC's), 16,000 skilled nursing homes (SNF's), 220 physician offices, 100,000 dental offices, 30,000 veterinary practices and 40,000 ambulances. Every business in the United States that handles controlled substances must comply with regulatory requirements. These regulatory requirements of controlled substances in medical environments have increased in complexity nearly as fast as the growth in controlled substance abuse and add greatly to the cost of healthcare organizations.

The largest and most sophisticated healthcare entities, hospitals, use expensive and sometimes outdated technology solutions to perform compliance. They rely on their large administrative staffs for this function. These organizations have primarily focused on solutions that maintain physical control of controlled substances through password protected cabinets and carts. While these solutions control access to controlled substances, they rely heavily on the employee to accurately count and input the quantity they have taken from the cabinet or cart. The employee does not have the comfort that the person before or after them was accurate in his/her count. Such an incident can put them and their organization at risk in the event of an incorrect count. In addition, each machine is connected to an in-house web-server. All reports are “self-service.” The servers depend upon purchase of software solutions for maintaining records and analyzing data for diversion. Inaccuracies must be reviewed, catalogued and stored. Smaller healthcare organizations and individual physicians do not have the luxuries of even these systems and instead rely on the honor system and manual records. The possibility of human errors and dishonest staff members increases the risk of personal liability for these small to mid-sized healthcare organizations and individual physicians. Devotion of personnel time to tracking and inventorying controlled substances and billing dispensed medications to the correct patients can also be costly. Moreover, on ambulances, medications and narcotics are kept for immediate treatment of the patient. The number of ambulances and their availability makes them prone to medication theft and diversion.

Besides the issue with controlled substances, currently there is no suitable system that can automatically keep track of free pharmaceutical samples in the environment of small healthcare organizations. In most cases, samples are just placed in a “sample closet” within the office by the pharmaceutical representatives or office staff. There is very little accounting regarding the incoming and outgoing of these samples. Sometimes expired medications are left inside these closets. In rare cases, even recalled medications may be on the shelves. Furthermore, very little of valuable data regarding the recipients of the medications are available to pharmaceutical companies.

Accordingly, an automated system and method for storing, dispensing, and inventorying medications, and for analyzing inventory data to detect diversion would be considered useful.

### SUMMARY

In accordance with one preferred embodiment, an access-controlled medication cabinet is provided. One or more such cabinets are connected to a central management computer. A central provider stocks cabinets with controlled medications. Users (e.g. doctors) gain physical access to contents of the cabinet by providing biometric identification. Human activities around and inside the cabinet are recorded on video. The video surveillance clips are transmitted to and stored on the central management computer. The system automatically tracks the transactions and inventories of all the cabinets and generates billing information accordingly through the use of RFID technology or the like. Analyzing inventory and use data on the central management computer enables in-time automatic reordering and other inventory optimizations. It also enables automatic detection of medication diversion. Records of transaction and surveillance clips are retained on the central management computer for a predetermined time as required by law or regulation for possible law enforcement investigation purposes, and may be sent to the government if required by law. Drug samples can also be distributed through the system in a similar fashion. Drug sample related use data are valuable to pharmaceutical companies, and can be turned over to them. Moreover, advertisements can be displayed on the front screen of the cabinet and/or another display screen connected to the cabinet.

According to one aspect, a method of maintaining a status of medical items used by a user of a customer of a service provider is provided, the method comprising: providing, from the service provider, a cabinet to the customer, wherein the cabinet stores medical items for dispensing to a patient; receiving at a computing system of the service provider, user access information of the user who withdrew one of the medical items from the cabinet, and medical item information on a type and quantity of the medical item which was withdrawn from the cabinet; and forwarding the user access information and the medical item information from the computing system to a medical regulatory authority.

According to another aspect, a method of maintaining a status of samples of medical items used by a user of a customer of a service provider is provided, the method comprising: storing a sample of a medical item by the service provider in a cabinet which has access restricted to authenticated users of the customer by the service provider; receiving at a computing system of the service provider, sample information on a type of the sample which was withdrawn from the cabinet by the user on behalf of a patient and patient information on attributes of the patient; and forwarding the sample information and the patient information from the computing system of the service provider to a manufacturer or representative of the manufacturer.

According to another aspect, a method of maintaining a status of medical items used by a user of a customer of a service provider is provided, the method comprising: providing, from the service provider, a cabinet to the customer, wherein the cabinet stores medical items for dispensing to a patient; and receiving at a computing system of the service provider, user access information of the user who withdrew one of the medical items from the cabinet, medical item information on a type and quantity of the medical item which was withdrawn from the cabinet, and location information regarding the time and location of the cabinet when the medical item was withdrawn from the cabinet.

According to another aspect, a method of maintaining a status of medical items used by a user of a customer of a service provider is provided, the method comprising: providing, from the service provider, a cabinet to the customer, wherein the cabinet stores medical items for dispensing to a patient; recording user access information of the user who has accessed the cabinet and recording information the user preparing a prescription for one of the medical items for the patient; and storing the recording information on a recording medium.

According to another aspect, a method of maintaining a status of medical items used by a user of a customer of a service provider is provided, the method comprising: providing, from the service provider, a cabinet to the customer, wherein the cabinet stores medical items for dispensing to a patient; using a first camera to take a first video of the user approaching the cabinet and activating a user access startup process to enable the user to access the cabinet; using a second camera to take a second video of the user accessing the cabinet and withdrawing one of the medical items from the cabinet; receiving at the cabinet, user access information of the user accessing the cabinet and acquiring medical information on a type and quantity of the medical item which was withdrawn from the cabinet; receiving at a computing system of the service provider, the first video, the second video, the user access information, and the medical item information; and storing at the computing system of the service provider, the first video, the second video, the user identification information, and the medical item information.

According to another aspect, a method of maintaining a status of medical items used by users of customers of a service provider is provided, the method comprising: providing, from the service provider, at least one cabinet with locks to each of the customers; stocking by the service provider medical items in each of the cabinets; receiving user identification information from the customer for each user of each customer; forwarding using a computing system of the service provider the user identification information to the corresponding cabinets for storage in the cabinets; using the cabinets to automatically record user access information, medical item information of the medical items withdrawn from the cabinets; and receiving the user access information and the medical item information at the computing system of the service provider for storage.

Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a front view of an access controlled medications storage and inventory control apparatus according to an embodiment of the present disclosure;

FIG. 2 is a perspective view of the access controlled medications storage and inventory control apparatus shown in FIG. 1;

FIG. 3 is a block diagram of a central control unit for use with the access controlled medication storage and inventory control apparatus as shown in FIG. 1;

FIG. 4 is a front view of an access controlled medications storage and inventory control apparatus in accordance with another embodiment;

FIG. 5 is a flowchart illustrating a method of registering a user and establishing permission settings for accessing the access controlled medication storage and inventory control cabinet of FIG. 1;

FIG. 6 is a flowchart illustrating a method of accessing items located within the access controlled medications storage and inventory control cabinet in accordance with an embodiment of the present disclosure;

FIG. 7 is a flowchart illustrating a method of performing product inventory utilizing the axis controlled medications storage and inventory control cabinet in accordance with an embodiment of the present disclosure;

FIGS. 8A and 8B are screen shots of a GUI interface of the access controlled medication storage and inventory control apparatus shown in FIG. 1, indicating how patient information can be entered;

FIG. 9 shows a storage, dispensing and inventory system which is utilized to store, dispense and keep inventory track of medical items;

FIG. 10 is a block diagram of a central management server of the storage, dispensing and inventory system shown in FIG. 9; and

FIG. 11 shows the various items of information that are present on the central control unit of the cabinet shown in FIG. 1 and on the central management server 50 shown in FIG. 9 operated by the service provider.

### DETAILED DESCRIPTION

Reference will now be made in detail to the present embodiments of the present invention, examples of which are illustrated in the accompanying drawings, wherein like reference numerals refer to the like elements throughout. The embodiments are described below in order to explain the present invention by referring to the figures.

Aspects of the present invention are directed to a system and method for dispensing and inventorying medications. It provides the medical practitioner with a total solution of controlled substance compliance. The system eliminates paperwork burden and significantly reduces the likelihood of controlled substance diversion.

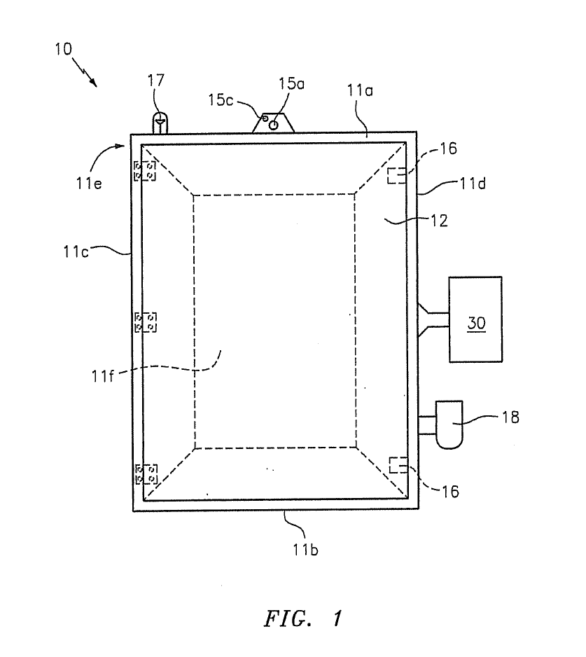

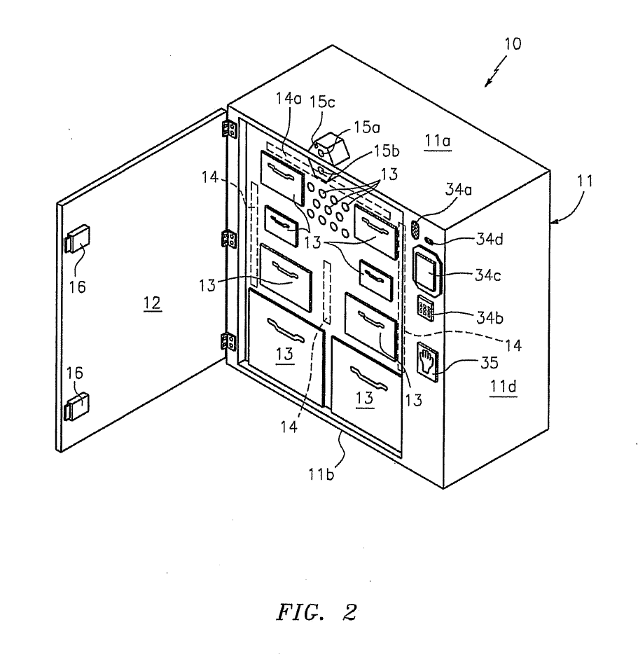

FIGS. 1 and 2 illustrate an access controlled medication storage and inventory control apparatus (e.g., cabinet) 10 which comprises a main body (frame) 11, a plurality of individual compartments 13, one or more inventory control modules 14 and a central control unit 30.

The main body 11 includes a top wall 11a, a bottom wall 11b, a pair of opposing side walls 11c and 11d, and a back wall 11e, thus forming an interior space 11f into which multiple compartments 13 are located. Each wall of the cabinet 10 may include a contagion suppression substance, such as an antimicrobial plastic coating, for example, to prevent the spread of bacteria, germs and viruses. An access door 12 is hingedly attached to one of the cabinet walls to provide access to the interior of the cabinet 11f.

A door 12 and the main body 11 further have one or more locks (i.e., electromagnetic, key, servo, etc.) 16 attached thereto to prevent unauthorized access to the interior of the cabinet. Each lock 16 is communicatively linked with a central control unit 30 to allow access to the cabinet only by authorized users.

An optional waste compartment 18 is secured to the outside of the cabinet frame 11. The waste compartment 18 can include steel, plastic or other secure enclosure having a one-way opening for allowing a user to deposit spent items such as vials, and syringes, for example. It is envisioned that the waste compartment would be used for completely used vials, but also for partially used dosages of the vials which still would have medication in them. The vials can be retrieved to determine whether vials which should still have some medication in them, actually do have the expected amount of medication therein.

The cabinet 10 may include mounting hardware such as screws, bolts, etc., to allow the cabinet 10 to be permanently secured to a fixed location. However, it is to be noted that the cabinet 10 may also be constructed in a fashion so as to be portable, such as for use in an ambulance. Although not illustrated, optional tamper switches can further be included to alert authorities when someone attempts to gain unapproved access to the cabinet 10. Such a feature can act as a protective mechanism for preventing the cabinet 10 from being moved, stolen or vandalized.

The cabinet 10 may also include a device tracking system 17, such as a global positioning satellite (GPS) or cellular location device capable of allowing authorities to track and locate the cabinet 10 in the event it is stolen.

The compartments 13 in FIG. 2 can be constructed to form any type of enclosure ranging from a drawer, tray, bin, shelf, syringe dispenser or other such enclosure, having a shape and dimension that is the same or different from other ones of the compartments 13.

In one embodiment, each compartment 13 can include an electronic locking mechanism (not illustrated) for securing the container and a closed position. Electronic lock of each compartment 13 can be independently controlled by the central control unit 30 to ensure that only authorized users have access to particular compartments 13.

As further illustrated in FIG. 2, the cabinet 10 also includes one or more inventory control modules 14, such as a radio frequency identification (RFID) reader, to provide an instantaneous count of the cabinet contents at all times. The inventory control module 14 receives data from RFID tags secured to each of the medical items stored within each compartment 13 compartment 13. As an item is removed, the inventory control module 14 captures a description of the item and reports are sent to the central control unit 30 for inventory purposes.

Instead of an RFID reader, a barcode scanner capable of reading a barcode or other identifying mark attached to each medical item may be used. In yet another embodiment, the inventory control module 14 can be an image recognition system (i.e., CCD, CMOS, digital, video, etc.) capable of identifying medical items by the markings or based on the physical shape of the particular medical item container. Such an image recognition system would work essentially the same as the barcode scanner, and would utilize product image descriptions stored within the memory of the central control unit 30.

As the cabinet 10 is designed to store valuable and/or regulated items, a first camera 15a is secured to the outside of the main body 11 to capture images of anyone attempting to access the cabinet 10 itself. A second camera 15b is secured to an inside of the main body 11 to provide a video record of each medical item removed by a particular user. The second camera 15b provides a visual record of all cabinet transactions and is useful for ensuring that employees follow company and/or regulatory guidelines when dealing with controlled substances.

The cabinet 10 may further include a motion detection sensor 15c capable of detecting motion near the cabinet 10. Upon detecting motion, the central control unit 30 activates the camera 15a and/or a user interface as described below.

A biometric identification unit 35 positively identifies the person attempting to access the cabinet 10. The biometric identification unit 35 maybe any of a number of commercially available devices capable of positively identifying a user based on the unique physical characteristic, such as a fingerprint, a handprint, a retinal and facial recognition system, etc. Instead of using biometrics, the cabinet 10 may use other secure identification devices, such as access card readers, key fob readers, a manual keypad and/or pass code systems.

The central control unit 30 is assigned to control the various functions of the access controlled medications storage and inventory control cabinet 10. The central control unit 30 can act as a gatekeeper for controlling access to the cabinet itself (via the biometric identification unit 35 and the locks 16) and/or to particular containers based on the users predetermined permission settings. Additionally the central control unit 30 can interface with the various inventory control modules 14 to store and report an accurate inventory of all medical items located within the cabinet 10.

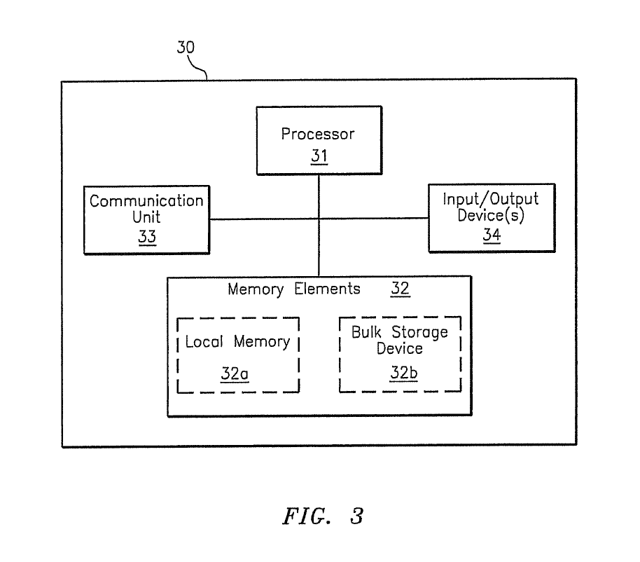

FIG. 3 illustrates a block diagram of the central control unit 30 that includes a processor 31 connected to an internal memory 32, a communications unit 33 and a plurality of input/output devices 34.

The processor 31 executes a program code stored in the internal memory 32 to allow the cabinet 10 to perform the functionality described herein. The internal memory 32 stores operating instructions in the form of program code for the processor 31 to execute. The internal memory 32 may include one or more physical memory devices such as a local memory 32a and/or one or more bulk storage devices 32b. Local memory 32a can refer to random access memory or other such memory device(s) generally use during actual execution of program code, whereas a bulk storage device can be implemented as a persistent data storage device. Additionally, the internal memory 32 can also include one or more cache memories that provide temporary storage of at least some program code to reduce the number of times program code is to be retrieved from the bulk storage device during execution.

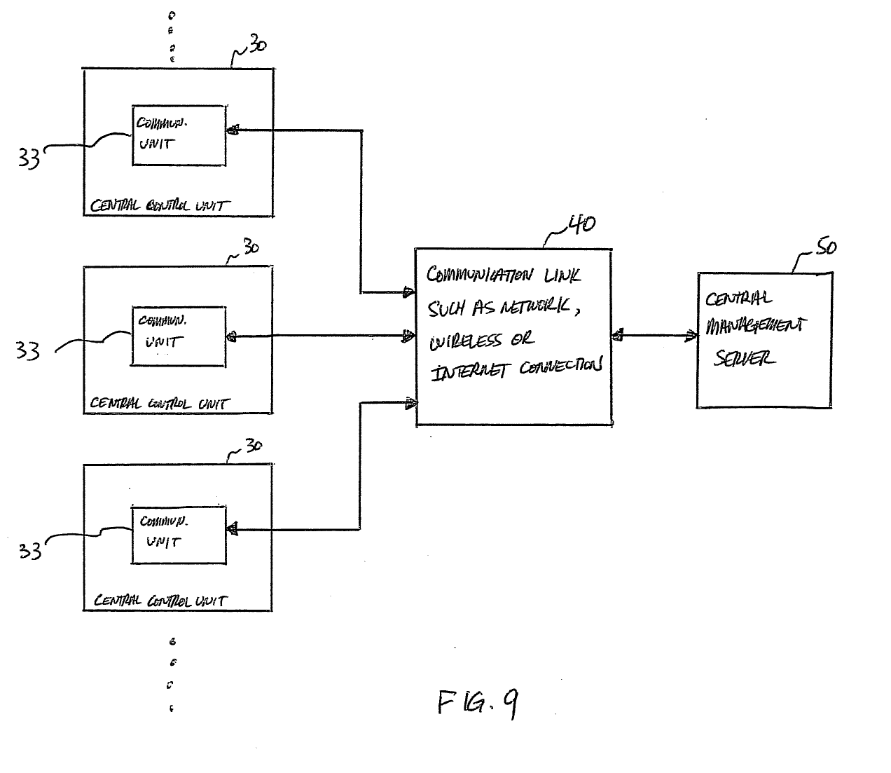

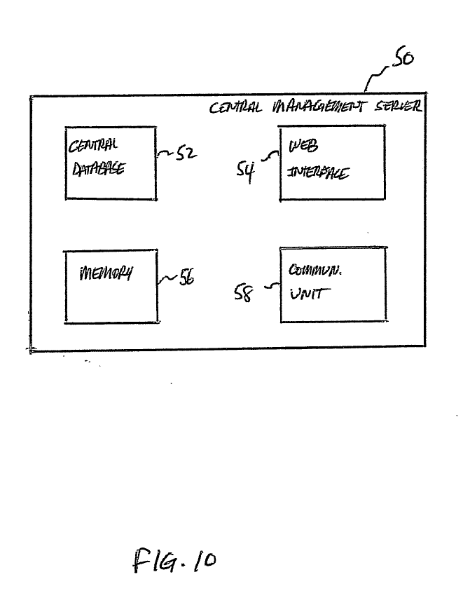

The communication unit 33 allows the central control unit 30 to communicate with external devices. The communication unit 33 can include any number of known devices such as the transmitter and receiver configured to send and receive wired and/or wireless communications i.e., radio frequency, infrared, Bluetooth, microwave, etc.), as well as network adapters (i.e., modems, cable modems, ethernet cards, WAN and LAN adapters). Consequently, the communication unit 33 provides two-way communication with other devices such as computer systems, remote printers, and/or remote storage devices through intervening private or public networks. The communication unit 33 can provide access to a communication link (e.g., ADSL, cable, Ethernet, fiber optic, etc.) or wireless (e.g., Wi-Fi, 3G, 4G, etc.) and a central management server 50 (described later and shown in FIGS. 9 and 10). Optionally, the data link may include access to the Internet, which in turn can provide a communication link to the central management server 50.

The communication unit 33 can include a USB port, this drive and/or writable CD drive configured to send and receive information with removable media devices ranging from a secure/writable CD, DVD, or an access controlled/password-protected flash drive, for example.

The input/output device(s) 34 may include a speaker 34a, one or more push buttons/keypads 34b, a display 34c and a microphone 34d.

In one embodiment, the display 34c includes a graphic user interface (GUI) capable of providing two-way communication with the user and the processor 31. The GUI interface 34c may include a color touch screen monitor to provide a menu of actions that a user can perform. As described below, the GUI interface 34c allows the user to sign for medications, gain access to the cabinet 10 itself, and display up-to-the-minute inventory of all medical items that have been removed from the cabinet 10 by the user. Alternatively when the cabinet 10 is not in use, the GUI interface 34c can display general information such as advertising, for example.

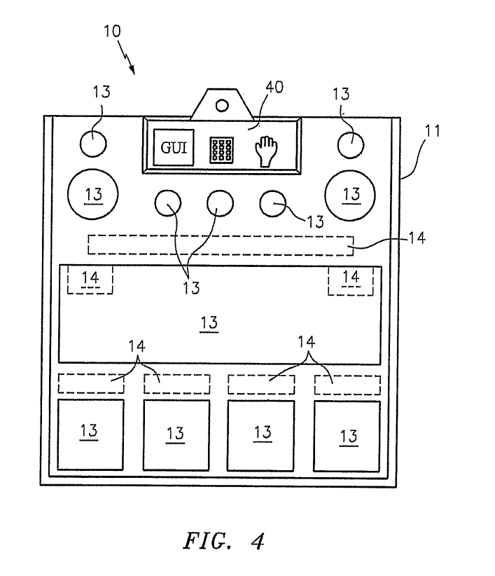

FIG. 4 illustrates an alternate embodiment of a cabinet 10 that includes an integrated identification and control unit 40. Integrated identification and control unit 40 can combine the functionality of the central control unit 30, the GUI interface 34c, one or more I/O devices 34, the biometric identification unit 35, the camera 15a and/or the motion sensor 15c into a single compact device. In one embodiment, the integrated identification and control unit 40 may comprise a tablet, for example, running a commercially available operating system configured to perform the various functionality described above.

According to an alternate embodiment, the cabinet 10 may further include a battery backup system to allow the cabinet 10 to function in the event that the main power to the device is lost.

As part of an initial contract or agreement between a service provider and a customer, the customer would fill out an informational application form for each user of the customer. The users may be a doctor, a nurse or other customer employee. Biometric information of each user is provided to the service provider along with other information, so that the service provider can conduct a background check and to download the biometric information and other identifying information of each user to the central control unit 30 via the central management server of the service provider (described later and shown in FIG. 9). This information may be referred to as user identification information, for example. Different levels of authorization can be provided for each user or for each type of user.

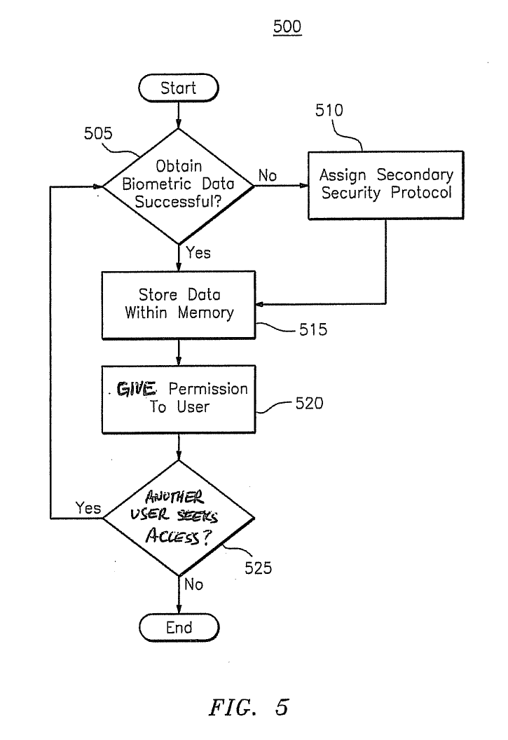

FIG. 5 is a flowchart illustrating a method 500 for enabling access by a user and confirming permission settings for accessing the cabinet 10.

The method begins by receiving credentials of a user for interfacing with the cabinet 10. Operation 505 captures biometric data such as a fingerprint, retinal, facial, or handprint readings from the user. Alternatively, the system can capture a voice sample from the user via the microphone 34d. At operation 510, the user provides a numeric code and/or physical evidence such as a key fob or access card having a registration unique to the particular user.

In operation 515, the user data is stored within the memory 32 for access by the processor 31. The method then proceeds to operation 520, where permission is given to the user. Each user can be assigned a unique or group permission settings which enables the user to access only those compartments 13 within the cabinet 10 that are pre-approved for the permission setting. For example, a doctor may have a permission setting that allows full access to each container, or a nurse may only have a permission setting that allows access to a particular compartment 13 compartment 13. In operation 525, additional users may seek access to the cabinet 10 by returning to operation 505, and otherwise the method terminates.

If in operation 505, the biometric information does not match that stored in the central control unit 30, the method proceeds to operation 510, where a user is allowed to enter a secondary security protocol (information) to have access to the cabinet 10.

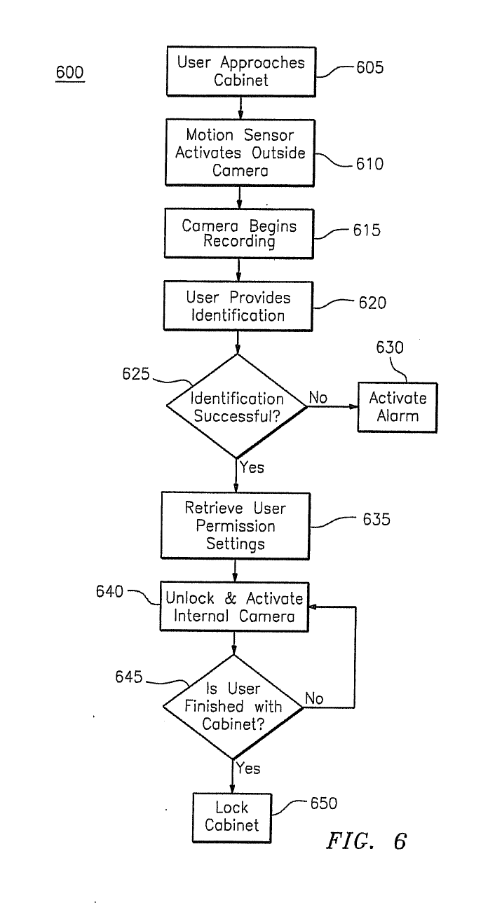

FIG. 6 is a flowchart illustrating a method 600 for accessing items located within the cabinet 10 described above. At operations 605 and 610, the user approaches the cabinet 10, and the motion detection sensor 15c activates the first camera 15a. At operation 615, the first camera 15a records the user's actions. Next, the user can provide identification to the cabinet 10 as per the operations 500-525 shown in FIG. 5. This information may be referred to as user access information, for example. Identification is established by utilizing the biometric identification unit 35, or one of the I/O devices 34 as assigned to the user during the registration process.

If the user is not recognized, the method proceeds to operation 630 where the user is allowed to repeat the identification procedure again, or may immediately activate an alarm (such as by emitting a sound through the speaker 34a and/or transmitting a distress signal via the communication unit 33).

If the user is recognized, the method proceeds to operation 635, where the processor 31 retrieves the user permission settings from the memory 32. Thus, operations 620-635 are analogous to operations 505-525. Upon receiving the permission settings, the method proceeds to operation 640, whether the processor 31 activates the internal camera 15b, unlocks the door 12 and unlocks the particular compartments 13 to which the user permission settings allow access.

Next, in operation 645, a determination is made as to whether the user is finished using the cabinet. The determination is based on the occurrence of a specific event or upon the passage of a predetermined period of time. For example, if the system detects that the cabinet door 12 has been close, the method proceeds to operation 650, where the system locks all containers and the door 12 and activates the internal camera 15b.

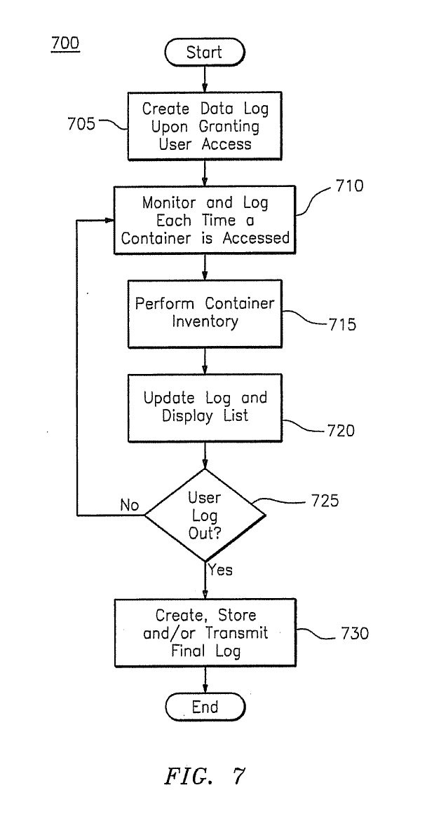

FIG. 7 is a flowchart illustrating a method 700 of performing product inventory utilizing the cabinet 10 described above.

At operation 705, a pre-registered user accesses the control panel (GUI interface 34c and/or keypads 34b) and selects a patient's name and/or patient number. Upon entry of this information, the system creates a data log in the memory 32 of the central control unit 30 that will include the video from the first and second cameras 15a and 15b, along with a date and time stamp. This log will remain open until the user logs out of the system.

At operation 710, the central control unit 30 monitors which compartments 13 are accessed by the user. Such a feature is accomplished by determining which of the electromagnetic locks are opened and noting the compartment 13 to which the lock is assigned.

Upon determining that the compartment 13 has been accessed, the method proceeds to operation 715, where the inventory control module(s) 14 associated with the accessed compartment 13 determines which, if any, medical items have been removed from the container 13. This information can also be compared with the visual record provided by the second (internal) camera 15b.

At operation 720, the removed items are noted in the data log, and displayed on the GUI interface 34c.

Operations 710-724 are repeatable until the user logs out at operation 725 of the cabinet 10 or the cabinet 10 automatically logs the user out. Upon determining that the user has logged out (or sensing inactivity for a predetermined period of time), the cabinet 10 locks the door 12 and all compartments 13.

Finally, the method proceeds to operation 730, where the final inventory count is stored within the memory 32 and/or transmitted to an external device via the communication unit 33. For example, the communication unit 33 can transmit the log containing a record of all anesthesiology items taken, the picture/video of the user and the time/date details to an actual device such as a hospital administrator or remote monitoring agency, for example. This information can then be relayed to other departments ranging from the patient's primary care physician, billing and/or other related parties. Further, the information can be relayed directly to a regulatory agency in accordance with voluntary procedures or issued regulations.

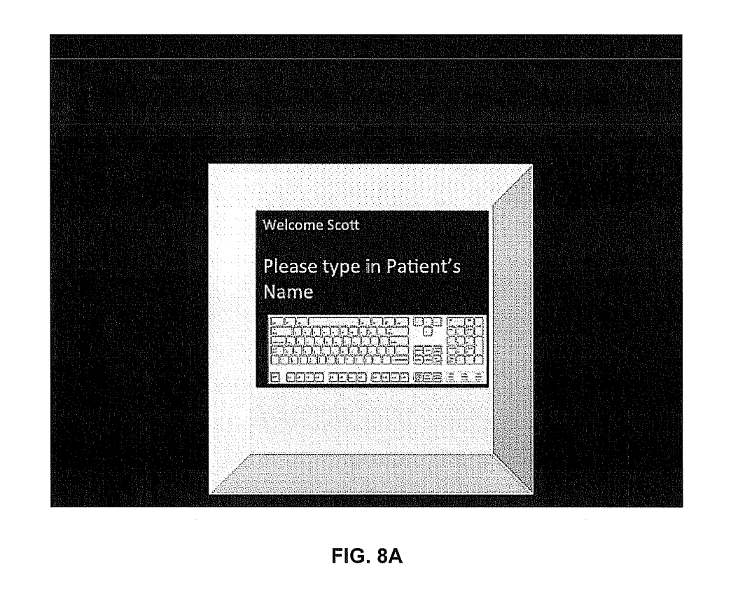

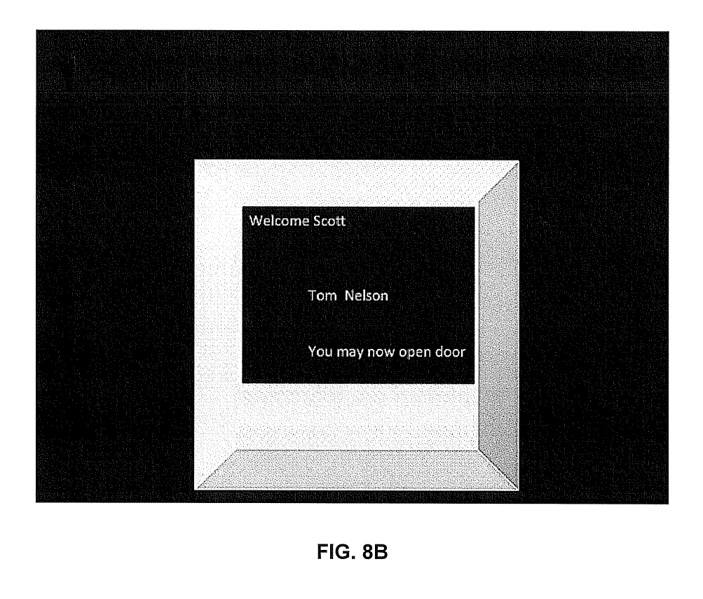

According to an embodiment of the present disclosure, at operation 705, the user is prompted to enter the patient's name or choose from a drop down list from the GUI interface 34c (see FIG. 8A). Once the patient data is properly entered, the door 12 opens (see FIG. 8B)

The cabinet 10 can be useful for doctors' offices which routinely deal with sample medication as well. The cabinet 10 can store and dispense medication samples while capturing a doctor's signature for each patient. In this regard, the doctor's signature can include the biometric data or other information used to access the cabinet 10. This information can then be stored in the memory 31, and be transmitted to a secure media card (i.e., secure flash drive) and/or pharmaceutical company/pharmacy, as required by various regulatory bodies.

FIG. 9 shows a storage, dispensing and inventory system 100 which is utilized to store, dispense and keep inventory track of medical items, including controlled or prescription items, and/or samples for one or a large number of access controlled medication storage and inventory control apparatuses (cabinets) 10. The storage, dispensing and inventory system comprises a plurality of the access controlled medication storage and inventory control apparatuses as described above, a communication link 40 and one or more central management servers (hereinafter referred to in the singular) 50.

The communication link 40 can be a network connection such as a WAN or LAN, a wireless connection such as Wi-Fi, 3G, 4G, etc., a connection through the Internet, or any communication link capable of connecting the communication unit 33 of the central control unit 30 of the cabinet 10 to the central management server 50. Preferably, the communication link is secure so as to prevent tampering by third parties.

With the storage, dispensing and inventory system 100, it is possible to provide secure equipment and cabinets, automated counting of medical items (both controlled and samples), and inventory management. Through its inventory management, the storage, dispensing and inventory system 100 will be able to meet and exceed any federal (including DEA), state and local regulations relating to the storing and dispensing of controlled/prescription substances and samples.

The cabinets 10 can be sold to customers or can be owned by the service provider and leased to the customers. The service provider owns the central management server 50.

As described above with reference to FIG. 6, when a user approaches the cabinet 10, the motion detector sensor 15c detects the motion, the first (outside) camera 15a is activated, and provides video (a video clip) of the user's approach. Either the GUI interface 34c changes to request information from the user, the keypad 34b is enabled to accept the user information or the biometric ID unit 35 is enabled to accept biometric measurements from the user. Once the user is confirmed, then the user is requested to enter patient information on a patient for whom a medical item (medicine) is needed. Input of the patient information can be input as set forth above and shown in FIGS. 8A and 8B. Namely, once a positive user identity is confirmed, the processor 31 prompts the user to enter patient information and/or account number on the GUI interface 34c. Alternatively, the processor 31 may prompt the user to select a patient from a list of patients on the touch screen of the GUI interface 34c.

Once the patient information is confirmed, namely the patient information is legitimate, the door 12 is unlocked and the second (inside) camera 15b is activated, thereby recording any activity of the user involving the inside of the cabinet 10 and withdrawal of any medical items from any of the compartments 13, and the particular compartment 13 which contains the medical item desired is unlocked. The inventory control module 14 tracks the remaining medical items in the compartment or the medical item that is removed, and associates the medical item with the patient and the user who took out the medical item. Upon an action from the user, the user may withdraw another medical item from another compartment 13, inform the cabinet 10 that the user is finished or the cabinet 10 determines that the user is finished based upon a predetermined amount of time of inactivity. The compartments 13 are locked and the door 12 is closed by the user, thereby shutting off the second (inside) camera 15b. The GUI interface 34c displays the medical item(s) removed during that session of the user by name and possibly by picture, such as through the use of icons identifying different types of medical items. The user is asked to confirm the removed contents. The door is then locked by the locks 16.

All of the data acquired by the cabinet 10, including the video from the first (outside) camera 15a, the second (inside) camera 15b, the user and patient information from the GUI interface 34c, the keypad 34b or the biometric ID unit 35 are sent to the central control unit 30, so that the central control unit 30 has the communication unit 33 send all of this information through the communication link 40 to the central management server 50. At least part of the data communication between the cabinet 10 and the central management server 50 can be in real-time instead of batched after conclusion of the whole transaction. The central management server 50 charges the customer's account accordingly, and the service provider can send a bill directly to each patient or provide the necessary data in an easily readable format to the customer so that the customer can bill the patient accordingly. If only medication samples are taken, no charge is made.

All of the medications stored in the compartments 13 are affixable with an RFID tag, provided by either a pharmaceutical manufacturer or the service provider. Alternatively, the RFID tags can contain serial numbers that are associated with more specific information about the medication in a regularly updated data table stored in a database of the central management server 50. Preferably, if such a data table is used, a local copy of the data table is maintained in the memory 32 of the cabinet 10 and updated from a central database (element 52 in FIG. 10) of the central management server 50 regularly. When a medical item is present in the cabinet 10, the information in its RFID tag is read, stored in the cabinet's 10 memory 32, and then transmitted to the central management server 50 to be stored in the central database 52. By taking a reading of all the RFID tags inside the cabinet, a current inventory at any given point in time can be determined. Preferably, a log of inventory changes is stored in the memory 32.

Thus, when a user takes a medical item out of a compartment 13, the event is reported to the central management server 50 of the service provider. The service provider is then able to: (1) charge customer accounts; (2) send emails of the transaction to the user and/or customer; (3) integrate the event with the customer's electronic medical records for notation on the appropriate patient's chart; (4) send the information to state/federal databases as required by state/federal law; (5) keep records of the transaction with a video clip for a determined amount of time as required by regulations; and (6) note which user took the medication. The event and related information can be transmitted from the communication unit 33 of the cabinet 10 to the central management server through encryption or over a secure and/or dedicated communication link 40.

As noted previously, the cabinet 10 further includes a device tracking system 17, such as a GPS module. Optionally the cabinet 10 may further include a wide-area wireless transmitter that is able to transmit GPS coordinates if the regular data link is susceptible to external disruption. Preferably, the GPS module and the wireless transmitter (if any) are backed up by an independent power source so that they are operative even when the external power source and the cabinet-wide backup power supply are both down. The GPS coordinates are transmitted to the central management server 50 periodically and automatically. Thus, in a situation where the cabinet 10 is used in a moving environment, such as on a cart movable throughout a doctor's office or hospital, or in an ambulance, the processor 31 is not only able to determine and associate the user, the patient and a corresponding medical item withdrawn from a compartment 13, but also able to determine the location of the cabinet 10 at any given time, as well as the location at which the compartment 13 from which the medical item was opened and closed. In the situation of use in an ambulance, it is then possible to determine whether the medical item was dispensed at the scene of an injury or illness or on the way to the hospital or some other location.

In accordance with another aspect of the present invention, the central management server 50 maintains the central database 52 in its memory and performs data aggregation and analysis and other managerial functions. One or more cabinets 10 are connected to the central management server 50 and exchange data with the corresponding communication units 33 from time to time. The central management server 50 timestamps all the transmissions received from the cabinets 10.

Every time the inventory of one of the cabinets 10 changes, the cabinet 10 transmits the updated inventory information to the central management server 50 through the communication unit 33. Logs of inventory changes of all cabinets 10 are kept in the central database (element 52 in FIG. 10) for at least a predetermined period of time. Logs are routinely analyzed to detect any aberration that may indicate diversion. In addition, statistical information regarding a specific medication (e.g., expiration, lot number, changes of use, peak usage times—days of week, months, seasons for specific medications), a specific patient (e.g., history, preference, etc.), or a specific cabinet can be mined by analyzing inventory logs. Such data analyses can be performed automatically and periodically. A warning is generated by the central management computer if any aberration is detected.

Information regarding individual patients and their accounts is stored in the central database 52. Every time a user removes medication for a patient and inputs the patient identity using the touch screen on the cabinet, the cabinet transmits the patient identity and the information about medications removed to the central management server 50. Patients' account information is changed accordingly. Bills can be generated for every patient based on his or her account information. Other patient-specific data reports including information about patient history and preferences can also be generated. With monitoring by the service provider, it is possible to determine if a patient has gone to other doctors to acquire the same medical items, and thus prevent abuse caused by doctor hopping patients (those that see multiple doctors to get multiple prescriptions for the same symptoms or illness).

With monitoring of the cabinets 10 by the central management server 50, it is possible for the service provider to watch out for and detect diversion. There are numerous algorithms available for detecting diversion. According to one algorithm, the central database 52 is accessed to determine how much of any medical item each doctor, nurse or other user withdraws from the cabinet over a predetermined period of time such as a month, compares the amount with similarly situated users who access that cabinet 10 or other cabinets 10 within a network of the customer, or compares the amount with similarly situated users in that field of medicine and/or within that area of the country. If the standard deviation is over a certain number, the user is flagged. Per the agreement between the customer and the service provider, the service provider is granted the right to investigate the user further or to report the situation to the appropriate authorities for them to investigate.

Another way for the service provider to monitor diversion is through the use of the waste compartment 18 shown in FIG. 1. As noted earlier, users can be put their completely used or partially used vials, such as syringes, that still have medication in them in the waste compartment 18. For example, the user may take a vial of fentanyl with 100 mcg in 2 cc, but only 1 cc or 50 mcg may be administered to the patient. The remainder is to be thrown in the waste compartment 18. Most systems call for a witness to see the handling of the waste. In small settings, this may not be practical. Also, if someone is trying to divert medications, they could simply replace the medication with water. With the waste compartment 18, the service provider can randomly test wasted vials later on. This procedure is an additional deterrent to diversion. Most personnel would be more hesitant to divert if they knew testing was occurring.

The sequence for wasting medication would be that the user would log on the touchscreen shown in FIGS. 8A and 8B what medication he/she was partially dosing or totally wasting in the event of a dropped vial. Then, the user would label the vial with the patient's name and put it into the waste compartment 18. The waste compartment has a rotating top like a cash drop so the user could put vials in but not take them out.

As shown in FIG. 10, the central management server 50 further includes a web interface 54 accessible by both system administrators and individual users with correct login information. It should be appreciated that instead of a server per se, any computing system capable of performing equivalent operations can be used to receive the information from the central control unit 30 of the cabinet 10 at the end of the service provider. Consistent with each web interface visitor's authorization, various kinds of data reports mentioned above can be requested and viewed, bills for the office or individual patients can be requested and viewed/downloaded, stored video clips can be viewed, and various system maintenance tasks can be performed. Alternatively, data reports and bills can be automatically and periodically generated and sent to users and/or patients, or made available on the web interface for immediate retrieval.

Optionally, relevant government agencies can also be given access to the web interface 54 to retrieve information they are entitled to under the law. Alternatively, a compilation of information due government agencies can be generated and sent to the government by system administrators through the web interface. In another alternative, the compilation of information can be generated and sent to the government automatically and periodically by the central management server 50.

When the inventory of one or more kinds of medications falls below a predetermined threshold in a particular cabinet, an order for restocking is automatically generated by the central management server 50. In addition, when some medication in a cabinet 10 is approaching expiration, an order for replacing the expiring medication is also automatically generated by the central management server 50. Further, the central management server 50 can generate an order indicating that there remains a large supply of a medical item in one of the cabinets 10 within a network of cabinets 10 of a customer of the service provider, so that the service provider can move some of the large supplied medical items from one cabinet 10 within the network to another cabinet 10, thereby delaying the need to acquire more of that type of medical item. Orders to optimize inventory across all the connected cabinets may also be generated, automatically utilizing medication-specific, patient-specific, and cabinet-specific information mentioned above.

According to one embodiment, the service provider provides the medical items on consignment and the medical items are thus owned by the service provider. It is the service provider that receives the orders that some medical items are in short supply, have expired or are nearing their expiration dates, or there is an uneven supply of medical items within the network of the customer, and the service provider goes to the customer physical site, refills the compartments 13 with medical items that are in short supply or expired, or moves medical items between compartments 13 and between cabinets 10 to even out supply or when medical items are approaching their expiration dates and can be relocated to cabinets 10 where the medical items are more likely to be used by the expiration date.

Demographic, preference, and possibly contact information for patients that use medication samples can be compiled automatically and sent to respective pharmaceutical companies by the central management server 50. Alternatively, the data can be made available for retrieval by pharmaceutical companies on the web interface 54.

Video clips recorded by the video cameras 15a and 15b included in the cabinets 10 are transmitted to and stored in a memory 56 of the central management server 50 each time a recording is completed. Alternatively, bandwidth permitting, video signals can be transmitted to the central management server 50 in real-time every time a video camera is activated. The video clips are retained on the central management computer for at least a predetermined about of time. The video clips can be made available to law enforcement agencies and respective users as required by law or by user request.

When the central management computer receives distress signals or GPS coordinates outside predetermined bounds, an alarm with detailed information is automatically generated by the central management server 50.

Another possible use of the storage, dispensing and inventory system 100 is to provide advertisements which can be loaded onto the central management server 50 and “pushed” through a communication unit 58 of the central management server 50 over the communication link 40 to the communication unit 33 of the cabinet 10 and then to the GUI interface 34c. Alternatively, the advertisements can be transferred into the memory 32 of the cabinet 10 via the data access port. The particulars of the advertisements can be distinguished and sent to particular cabinets 10 according to the type of customer using the cabinet or according to an agreement between the customer and the service provider. The advertisements can also be transmitted via a fixed line or wireless communication from the cabinet 10 to the waiting area of the doctor's office or hospital or an examination area of the doctor's office or hospital.

In accordance with yet another aspect of the invention, a method is provided for dispensing and inventorying medications. Instead of withdrawing a prescribed medical item, if the user intends to take out a medication sample, he or she can input the patient's e-mail address if the patient consents. If the user intends to take out a medication sample, he or she should press a “sample” button on the GUI interface 34c and select the specific sample he or she is taking on the touch screen or keypad. By pressing the “sample” button, the user will satisfy the request system required by pharmaceutical regulations for samples.

The service provider acts as an agent of a contracted pharmaceutical company to stock their samples within the cabinet 10 of a customer. Since it is an open shelf system, manufacturers (pharmaceutical company) will not have to change their FDA approved packaging for samples to be placed in the cabinet 10. The pharmaceutical company will be supplied data regarding the sample usage.

The customer receives periodical bills generated automatically by the central management server 50 reflecting medication usage and charges during the billing period. The customer may also elect to receive data analysis reports pertaining to his or her office/organization compiled by the central management server 50. Patient-specific reports may also be included. Patient history and preferences are included in such patient-specific reports. Alternatively, the customer may request such reports through the web interface 54 if and when he or she wants these reports. The customer may also request bills for individual patients. If the customer uses an electronic medical record system compatible with the system described in the embodiment, the customer may also opt to integrate patient-specific data mentioned above into the patient's chart in an electronic medical record system.

Optionally, a video recording can be made by the first (outside) or second (internal) cameras 15a or 15b of the cabinet 10 to verify prescriptions. The recording can be transferred to external storage media, such as a memory stick, via the data access port, or sent to the central management server 50. The external storage media can then be taken to a pharmacy to serve to verify the prescription. Alternatively, the recording can also be transmitted over the communication link 40 directly to a pharmacy to verify the prescription. Still alternatively, the recording can be transmitted over the communication link 40 to the central management server 50, which sends the recording of the prescription to the pharmacy. If there is an authorization agreement between the service provider and the pharmacy, then the central management server 50 could determine whether the user is authorized and forward the recording or an authorization to the pharmacy.

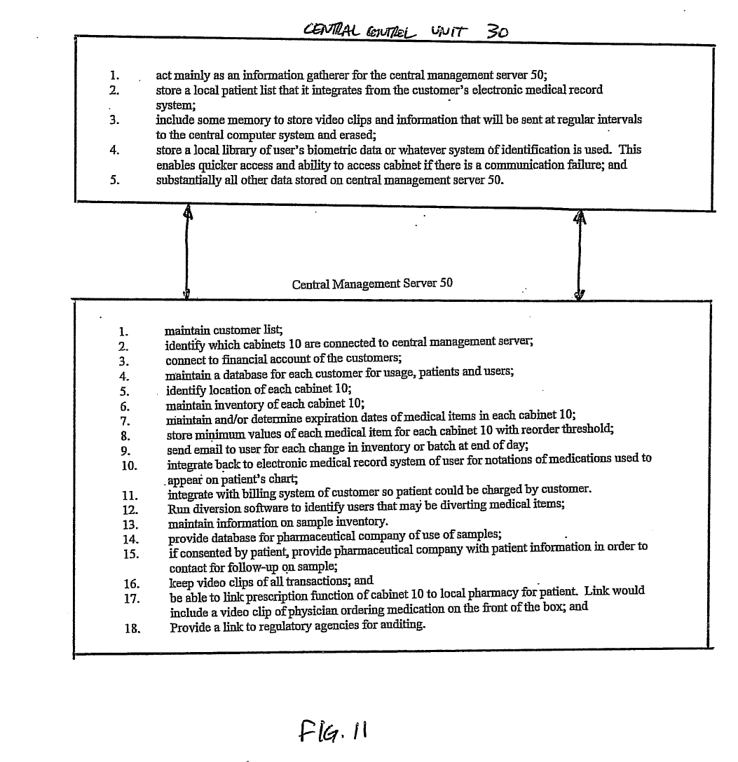

FIG. 11 shows the various items of information that are present on the central control unit 30 of the cabinet 10 and on the central management server 50 operated by the service provider.

The central control unit 30 of each cabinet 10 acts mainly as an information gatherer for the central management server 50. The central control unit 30 has the capacity to store all of the information necessary to carry out its operations as described above. The central control stores the following information, but is not limited to such information, and can be programmed to perform additional operations, including some performed by the central management system 50.

The central control unit 30: stores a local patient list that it integrates from the electronic medical record system of the service provider; has some memory to store video clips and information that will be sent at regular intervals to the central management server 50 and erased or will be sent in essentially real-time; and has a local library of each user's biometric data or whatever system of identification is used. The local library makes for quicker access and ability to access the cabinet 10 if there is a communication failure between the central control unit 30 and the central management server 50.

As a general matter, substantially all other data is stored on the central management server 50. The central control unit 30 has the capacity to store all of the information necessary to carry out its operations as described above. The central management server 50 stores the following information, but is not limited to such information, and can be programmed to perform additional operations, including some performed by the central control unit 30.

The central management server 50: maintains a list of customers of the service provider; identify which cabinets 10 are connected to the central management server; connects to financial account information of the customers; maintains a database for each customer regarding usage, patients and users; identifies the location of each cabinet 10; stores the inventory of each cabinet 10; maintains and/or determines expiration dates of medical items in each cabinet 10; stores set minimum values of each medical item for each cabinet as a reorder threshold; sends an email to each customer for each change in inventory or batch at the end of a day; integrates back to an electronic medical record system of the customer for notations of medications used to appear on a patient's medical chart; integrates with a customer billing system so each patient could be charged by the customer; runs diversion software to identify users that may be diverting medical items; keeps track of sample inventory; provides a database for a pharmaceutical company on the use of the pharmaceutical companies' samples; if consented to by a patient; provides the pharmaceutical company with patient information in order to contact for follow-up on the sample; keeps video clips of all transactions; has the ability to link a prescription function of the cabinet 10 to a local pharmacy for a patient, wherein the link would include a video clip of a physician ordering medical items on the front of the cabinet 10; and provides a link to regulatory agencies for auditing purposes.

As set forth in the foregoing, described are a system and a method of securely storing, dispensing, and inventorying medications and samples. The system and method allow a service provider to provide a remote secure way to monitor medications and samples dispensed by its customers, enable restocking of depleted inventory, enable easy and automatic billing to patients, promote samples from a pharmaceutical company, effect compliance with federal and/or state regulatory authorities, among other benefits. The system and method relieve the customers of implementing costly, time-consuming and personnel time draining procedures which the customers are ill-equipped to manage or afford.

Although a few embodiments of the present invention have been shown and described, it would be appreciated by those skilled in the art that changes may be made in this embodiment without departing from the principles and spirit of the invention, the scope of which is defined in the claims and their equivalents.

What is claimed is:

1. A method of maintaining a status of medical items used by a user of a customer of a service provider, the method comprising:
providing, from the service provider, a cabinet to the customer, wherein the cabinet stores medical items for dispensing to a patient;
receiving at a computing system of the service provider, user access information of the user who withdrew one of the medical items from the cabinet, and medical item information on a type and quantity of the medical item which was withdrawn from the cabinet; and
forwarding the user access information and the medical item information from the computing system to a medical regulatory authority.

2. A method of maintaining a status of samples of medical items used by a user of a customer of a service provider, the method comprising:
storing a sample of a medical item by the service provider in a cabinet which has access restricted to authenticated users of the customer by the service provider;
receiving at a computing system of the service provider, sample information on a type of the sample which was withdrawn from the cabinet by the user on behalf of a patient and patient information on attributes of the patient; and
forwarding the sample information and the patient information from the computing system of the service provider to a manufacturer or representative of the manufacturer.

3. The method of claim 2, further comprising:
if the patient has provided consent to allow contact information to be provided to the manufacturer, receiving the contact information at the computing system of the service provider and forwarding the contact information along with the sample information and the patient information from the computing system to the manufacturer.

4. The method of claim 2, further comprising:
retrieving other sample information provided by the manufacturer or representative of the manufacturer by the computing system; and
forwarding the other sample information to a graphical user interface of the cabinet.

5. The method of claim 2, further comprising:
retrieving other sample information provided by the manufacturer or representative of the manufacturer by the computing system; and
forwarding the other sample information to a display device in a waiting or examination area of the customer.

6. The method of claim 2, further comprising:
receiving a request from the user at the cabinet for the sample in accordance with regulations set up by medical regulatory authority.

7. A method of maintaining a status of medical items used by a user of a customer of a service provider, the method comprising:
providing, from the service provider, a cabinet to the customer, wherein the cabinet stores medical items for dispensing to a patient; and
receiving at a computing system of the service provider, user access information of the user who withdrew one of the medical items from the cabinet, medical item information on a type and quantity of the medical item which was withdrawn from the cabinet, and location information regarding the time and location of the cabinet when the medical item was withdrawn from the cabinet.

8. A method of maintaining a status of medical items used by a user of a customer of a service provider, the method comprising:
providing, from the service provider, a cabinet to the customer, wherein the cabinet stores medical items for dispensing to a patient;
recording user access information of the user who has accessed the cabinet and recording information of the user preparing a prescription for one of the medical items for the patient; and
storing the recording information on a recording medium.

9. The method of claim 8, wherein the recording information is video of the user preparing the prescription.

10. The method of claim 9, further comprising:
receiving at a computer system of the service provider, the user access information and the video; and
forwarding the video of the prescription from the computing system to a pharmacy for filling.

11. The method of claim 10, further comprising:
checking, using the computing system, whether the user has authorization to write the prescription based upon the user access information prior to forwarding the video of the prescription from the computing system to the pharmacy.

12. The method of claim 10, wherein the recording medium is a memory stick connected to an access port of the cabinet.

13. A method of maintaining a status of medical items used by a user of a customer of a service provider, the method comprising:
providing, from the service provider, a cabinet to the customer, wherein the cabinet stores medical items for dispensing to a patient;
using a first camera to take a first video of the user approaching the cabinet and activating a user access startup process to enable the user to access the cabinet;
using a second camera to take a second video of the user accessing the cabinet and withdrawing one of the medical items from the cabinet;
receiving at the cabinet, user access information of the user accessing the cabinet and acquiring medical information on a type and quantity of the medical item which was withdrawn from the cabinet;
receiving at a computing system of the service provider, the first video, the second video, the user access information, and the medical item information; and
storing at the computing system of the service provider, the first video, the second video, the user access information, and the medical item information.

14. The method of claim 13, further comprising:
in response to the first video, the second video, the user identification information and medical item information, using the computing system to:
charge an account of the customer corresponding to the patient;
send an email from the computing system to the cabinet indicating access by the user;
input the medical item information to a medical chart of the patient; and
send the corresponding information to a regulatory authority.

15. The method of claim 13, further comprising:
in response to the first video, the second video, the user identification information and medical item information, using the computing system to:
send a bill directly to the patient;
send an email from the computing system to the cabinet indicating access by the user;
input the medical item information to a medical chart of the patient; and
send the corresponding information to a regulatory authority.

16. The method of claim 13, further comprising:
assessing whether the first video, the second video, the user identification information and the medical item information match.

17. A method of maintaining a status of medical items used by users of customers of a service provider, the method comprising:
providing, from the service provider, at least one cabinet with locks to each of the customers;
stocking by the service provider medical items in each of the cabinets;
receiving user identification information from the customer for each user of each customer;
forwarding using a computing system of the service provider the user identification information to the corresponding cabinets for storage in the cabinets;
using the cabinets to automatically record user access information, medical item information of the medical items withdrawn from the cabinets; and
receiving the user access information and the medical item information at the computing system of the service provider for storage.

18. The method of claim 17, further comprising:
running a diversion program on the user access information and the medical item information at the computing system to determine if one of the users is improperly diverting medical items.

19. The method of claim 17, further comprising:
determining, using the computing system, whether any of the medical items in any of the cabinets is below a threshold level; and
restocking, by the service provider, the medical items which are below the threshold level based upon the determination.

20. The method of claim 17, further comprising:
performing a background check on each user by the service provider in response to the user identification information received from the customer and prior to the computer system forwarding the user identification system to the corresponding cabinets.

21. The method of claim 17, further comprising:
keeping track of expiration date, lot number, changes of usage and peak usage times for the medical items which are stored in and withdrawn from the cabinet according to the medical item information, using the computing system.

22. The method of claim 17, further comprising:
using the cabinets to record patient information entered by the user and corresponding to the medical item information withdrawn by the users; and
receiving the patient identification at the computing system from the cabinets.

23. The method of claim 17, wherein the medical item information includes an indication of partial dispensing of a dosage of medicine in the withdrawn medical items, the method further comprising:
comparing, by the service provider, the medical items deposited into waste compartments of corresponding ones of the cabinets with the medical item information on the partially dispensed dosages from the medical items for assessment whether diversion has occurred.

24. A non-transitory computer-readable medium having computer executable code stored thereon, the code, operated by a service provider, for maintaining a status of medical items used by a user of a customer of a service provider, the code comprising:
providing, from the service provider, a cabinet to the customer, wherein the cabinet stores medical items for dispensing to a patient;
code to receive user access information of a user who withdrew one of medical items stored in a cabinet for dispensing to a patient, and medical item information on a type and quantity of the medical item which was withdrawn from the cabinet; and
code to forward the user access information and the medical item information to a medical regulatory authority.

25. A non-transitory computer-readable medium having computer readable code stored thereon, the code for maintaining a status of samples of medical items used by a user of a customer of a service provider, the code comprising:
code to receive sample information on a type of a sample which was withdrawn from a cabinet by a user on behalf of a patient and patient information on attributes of the patient; and
code to forward the sample information and the patient information to a manufacturer or representative of the manufacturer.

26. A non-transitory computer-readable medium having computer readable code stored thereon, the code for maintaining a status of medical items used by a user of a customer of a service provider, the code comprising:
code to receive user access information of a user who withdrew one of medical items from a cabinet;
code to receive medical item information on a type and quantity of the medical item which was withdrawn from the cabinet, and code to receive location information regarding the time and location of the cabinet when the medical item was withdrawn from the cabinet; and
code to process the user access information, the medical item information and the location information.

27. A non-transitory computer-readable medium having computer readable code stored thereon, the code for maintaining a status of medical items used by a user of a customer of a service provider, the code comprising:
code to record user access information of the user who has accessed a cabinet in which medical items for dispensing to a patient are stored;
code to record video of the user preparing a prescription for one of the medical items for the patient; and
code to forward the user access information and the video to a pharmacy for filling.

28. A non-transitory computer-readable medium having computer readable code stored thereon, the code for maintaining a status of medical items used by a user of a customer of a service provider, the code comprising:
providing, from the service provider, a cabinet to the customer, wherein the cabinet stores medical items for dispensing to a patient;
code to receive a first video from a first camera of a user approaching a cabinet which stores medical items for dispensing to a patent, and to activate a user access startup process to enable the user to access the cabinet;
code to receive a second video from a second camera of the user accessing the cabinet and withdrawing one of the medical items from the cabinet;
code to receive user access information of the user accessing the cabinet and acquiring medical information on a type and quantity of the medical item which was withdrawn from the cabinet; and
code to store the first video, the second video, the user access information, and the medical item information.

29. A non-transitory computer-readable medium having computer readable code stored thereon, the code for maintaining a status of medical items used by users of customers of a service provider, the code comprising:
code to determine which medical items are to be stocked by a service provider in each of cabinets of the customers;
code to input user identification information received from the customer for each user of each customer;
code to forward user identification information to the corresponding cabinets for storage in the cabinets;
code to receive user access information acquired by inputs from the user to access the cabinets, and to receive medical item information of the medical items withdrawn by the users; and
code to alert the service provider on inventory of the medical items based upon the received medical item information.
